(19) United States
(12) Patent Application Publication
(10) Pub. No.: US 2024/0023543 A1
(43) Pub. Date: Jan. 25, 2024

(54) METHOD AND APPARATUS FOR REDUCING EXCESS PRESSURE IN ISOCHORIC SYSTEMS

(71) Applicant: BioChoric, Inc., Bozeman, MT (US)

(72) Inventors: Matthew J. Powell-Palm, Bozeman, MT (US); Anthony N. Consiglio, Berkeley, CA (US)

(21) Appl. No.: 18/222,436

(22) Filed: Jul. 15, 2023

Related U.S. Application Data
(60) Provisional application No. 63/390,688, filed on Jul. 20, 2022.

(51) Int. Cl. A01N 1/02

(52) U.S. Cl. CPC A01N 1/0284; A01N 1/0221; A01N 1/0242

(57) ABSTRACT

A method for reducing excess pressure in isochoric systems that involves providing a rigid and sealable master container, placing a primary subsystem comprised of biological matter into the master container, placing a secondary subsystem into the master container, removing bulk gas phase from the master container, sealing the master container, cooling the master container to a desired sub-0° Centigrade storage temperature, maintaining the master container at the storage temperature for a storage period, warming the master container to a temperature that is greater than the equilibrium melting point of the primary subsystem, unsealing the master container, and removing the biological matter from the master container. The secondary subsystem is a liquid that is immiscible with water and has a positive coefficient of thermal expansion that is greater in absolute magnitude than the coefficient of thermal expansion of water at sub-0° Centigrade temperatures. An apparatus for performing the foregoing method steps.

### CROSS-REFERENCE TO RELATED APPLICATION

Pursuant to 35 U.S.C. § 119(e), this application claims the benefit of U.S. Patent Application No. 63/390,688, filed on Jul. 20, 2022, the contents of which are incorporated herein by reference.

### BACKGROUND OF THE INVENTION

### 1. Field of the Invention

The present invention relates generally to methods and systems for preserving biological matter, and more particularly, to a method and apparatus for preventing pressure-based damage to biological matter preserved in an isochoric system at sub-zero Centigrade temperatures by reducing the excess positive or negative pressures created by thermo-volumetric changes in the contents of the system.

### 2. Description of the Related Art

There is growing interest in the use of isochoric (constant-volume) systems for the low-temperature preservation of biological matter [1]. These systems leverage the unique thermodynamic effects of isochoric conditions to prevent aqueous solutions bearing biological contents from freezing undesirably and damaging said contents, while maintaining the contents at sufficiently low temperatures to arrest metabolism and extend the period for which they may be preserved. Among isochoric preservation techniques, isochoric supercooling [2], [3] and isochoric vitrification [4] do not involve a first-order phase change; i.e., they involve maintaining the contained aqueous contents (including aqueous contents within any contained biological matter) in a single homogeneous phase. During isochoric supercooling, which generally proceeds in the temperature range 0° C. to −40° C., these aqueous contents are liquid. During isochoric vitrification, which generally proceeds in the temperature range −80° C. to −273° C., the aqueous contents are first liquid and then vitreous glass [5].

All materials either expand or contract upon change in temperature; i.e., all materials have finite, non-zero coefficients of thermal expansion (although select materials may have a thermal expansion coefficient of zero at a singular value in temperature at which the thermal expansion value changes from positive to negative). Thus, when confined in a rigid constant-volume isochoric system, any changes in temperature will cause the pressure experienced within the system to either increase (if the contents within tend to significantly expand) or decrease (if the contents within tend to significantly contract). In an isochoric system, the resulting pressures are a function only of temperature, and the relationship between temperature and pressure is an immutable function of the thermo-volumetric properties of the contained contents.

The term “thermo-volumetric,” as applied to the properties or changes of a material, describes how a material will change in volume in response to a change in temperature and pressure. The specific properties dominating the relationship between temperature and pressure in an isochoric system are the thermal expansivity, or the degree to which the material changes in volume with temperature at a given pressure, and the compressibility, or the degree to which the material changes in volume with pressure at a given temperature. The absolute change in volume of a material is also proportional to its initial volume. It is important to note that any pressures that emerge in single-phase isochoric systems are driven by thermo-volumetric changes in the materials present, as opposed to expansion or contraction of additional materials produced via phase change (such as ice), as is often encountered during conventional isochoric freezing [6], [7].

For biological specimens, prolonged exposure to pressures either greater than or less than pressures experienced under normal homeostatic conditions may prove to be deleterious [8], [9]; therefore, during isochoric cryopreservation protocols, it is of great interest to affect the relationship between temperature and pressure experienced within the system in order to ensure that, at the desired preservation temperature, the pressure rests at the desired value. In other words, it is desirable to reduce excess pressure, be it positive or negative, that may be produced by thermo-volumetric changes in the interior contents.

The temperature-pressure relationship for systems in which two-phase liquid-ice configurations are present (referred to commonly as “isochoric freezing”) has been well characterized and leveraged in a number of previous devices [6]. Methods of affecting the pressure-temperature relationship and reducing excess pressures for single-phase supercooled or vitrified isochoric systems have not been previously reported, however.

Various dissimilar aspects of isochoric cryopreservation have been taught in prior art. Rubinsky and Szobota teach how isochoric conditions may be used to stabilize isochoric supercooling against unintended homogeneous ice nucleation (U.S. Patent Application Pub. No. 20070042337). Powell-Palm and Rubinsky teach how isochoric conditions may be used to stabilize isochoric supercooling against unintended heterogeneous ice nucleation and how a device can be used to monitor the nucleation of ice from a supercooled system using a pressure transducer (International Patent Application No. PCT/US21/12863). Rubinsky el al, teach how a multi-step temperature cycle may be used to achieve vitrification in isochoric systems, and how monitoring the pressure for the large increases associated with ice formation can be used to verify the success of vitrification (U.S. Patent Application Pub. No. 20200178518). All of these disclosures, while pertaining to many valuable aspects of isochoric supercooling and isochoric vitrification, ignore the potentially deleterious excess pressures (positive or negative) that may be created by simple thermo-volumetric changes in the contents within an isochoric chamber (as opposed to phase changes), and none provides a means for reducing this excess pressure.

### Objects of the Present Invention

The present invention is directed to a method and an apparatus for reducing excess positive or negative pressure during isochoric preservation. The present invention is relevant to the preservation of biological matter by isochoric vitrification and isochoric supercooling. More specifically, the present invention provides a method and an apparatus for reducing the excess positive or negative pressure applied to biological matter in an isochoric system by preserving the biological matter in a supercooled state at temperatures lower than the equilibrium melting point of the biological matter and the solution in which it is kept. The present invention also provides a method and an apparatus for reducing the excess positive or negative pressure applied to biological matter in an isochoric system by preserving the biological matter in a vitrified or partially vitrified state at temperatures lower than the glass transition temperature of the biological matter and the solution in which it is kept. In both cases, the present invention reduces the excess positive or negative pressure applied to the biological matter by reducing the net thermo-volumetric change of the different elements stored within an isochoric container at the desired storage temperature.

### BRIEF SUMMARY OF THE INVENTION

The present invention is a method for reducing excess pressure in isochoric systems comprising: providing a rigid and sealable master container; placing a primary subsystem comprised of biological matter into the master container; placing a secondary subsystem into the master container, wherein water has a negative coefficient of thermal expansion at sub-0° Centigrade temperatures; and wherein the secondary subsystem is a liquid that is immiscible with water and has a positive coefficient of thermal expansion that is greater in absolute magnitude than the coefficient of thermal expansion of water; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container, wherein water has a negative coefficient of thermal expansion at sub-0° Centigrade temperatures; and wherein the secondary subsystem is a liquid that is immiscible with water and has a negative coefficient of thermal expansion that is greater in absolute magnitude than the coefficient of thermal expansion of water; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container; wherein water has a negative coefficient of thermal expansion at sub-0° Centigrade temperatures; and wherein the secondary subsystem is a liquid that is immiscible with water and has a positive coefficient of thermal expansion that is lesser in absolute magnitude than the coefficient of thermal expansion of water; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container; wherein water has a negative coefficient of thermal expansion at sub-0° Centigrade temperatures; and wherein the secondary subsystem is a liquid that is immiscible with water and has a negative coefficient of thermal expansion that is lesser in absolute magnitude than the coefficient of thermal expansion of water, removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container; wherein water has a negative coefficient of thermal expansion at sub-0° Centigrade temperatures; and wherein the secondary subsystem is a solid that is immiscible with water and has a positive coefficient of thermal expansion that is greater in absolute magnitude than the coefficient of thermal expansion of water; removing bulk gas phase from the master container, sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container, placing a secondary subsystem into the master container; wherein water has a negative coefficient of thermal expansion at sub-0° Centigrade temperatures; and wherein the secondary subsystem is a solid that is immiscible with water and has a negative coefficient of thermal expansion that is greater in absolute magnitude than the coefficient of thermal expansion of water; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container, wherein water has a negative coefficient of thermal expansion at sub-0° Centigrade temperatures; and wherein the secondary subsystem is a solid that is immiscible with water and has a positive coefficient of thermal expansion that is lesser in absolute magnitude than the coefficient of thermal expansion of water; removing bulk gas phase from the master container, sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container; wherein water has a negative coefficient of thermal expansion at sub-0° Centigrade temperatures; and wherein the secondary subsystem is a solid that is immiscible with water and has a negative coefficient of thermal expansion that is lesser in absolute magnitude than the coefficient of thermal expansion of water; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container; wherein water has a negative coefficient of thermal expansion at sub-0° Centigrade temperatures; wherein the secondary subsystem is a liquid that is miscible with water and has a positive coefficient of thermal expansion that is greater in absolute magnitude than the coefficient of thermal expansion of water; and wherein the liquid is separated from the primary subsystem by a mass-impermeable barrier; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container; wherein water has a negative coefficient of thermal expansion at sub-0° Centigrade temperatures; wherein the secondary subsystem is a liquid that is immiscible with water and has a negative coefficient of thermal expansion that is greater in absolute magnitude than the coefficient of thermal expansion of water; and wherein the liquid is separated from the primary subsystem by a mass-impermeable barrier; removing bulk gas phase from the master container; sealing the master container, cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and scalable master container; placing a primary subsystem comprised of biological matter into the master container; placing a secondary subsystem into the master container; wherein water has a negative coefficient of thermal expansion at sub-0° Centigrade temperatures; wherein the secondary subsystem is a liquid that is immiscible with water and has a positive coefficient of thermal expansion that is lesser in absolute magnitude than the coefficient of thermal expansion of water, and wherein the liquid is separated from the primary subsystem by a mass-impermeable barrier; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In an alternate embodiment, the present invention is a method for reducing excess pressure in isochoric systems comprising: providing a rigid and sealable master container; placing a primary subsystem comprised of biological matter into the master container; placing a secondary subsystem into the master container; wherein water has a negative coefficient of thermal expansion at sub-0° Centigrade temperatures; wherein the secondary subsystem is a liquid that is immiscible with water and has a negative coefficient of thermal expansion that is lesser in absolute magnitude than the coefficient of thermal expansion of water; and wherein the liquid is separated from the primary subsystem by a mass-impermeable barrier; removing bulk gas phase from the master container; sealing the master container; cooling the master container to a desired sub-0° Centigrade storage temperature; maintaining the master container at the desired storage temperature for a desired storage period; wherein the primary subsystem has an equilibrium melting point, warming the master container to a temperature that is greater than the equilibrium melting point of the primary subsystem; unsealing the master container; and removing the biological matter from the master container.

In a preferred embodiment, the secondary subsystem is comprised of the group consisting of mineral oil, vegetable oil, silicone oil, and perfluorocarbon. In an alternate embodiment, the secondary subsystem is comprised of pure water.

In a preferred embodiment, the present invention further comprises the step of: providing a mechanical element that is configured to increase and decrease volume of the master container. In a preferred embodiment, the master container is comprised of a material that possesses a coefficient of thermal expansion that is higher than that of grade 5 titanium.

In one embodiment, the present invention further comprises the step of: combining at least one primary subsystem and more than one secondary subsystem within the same master container. In another embodiment, the present invention further comprises the step of: combining more than one primary subsystem and at least one secondary subsystem within the same master container. In yet another embodiment, the present invention further comprises the step of: combining more than one primary subsystem and more than one secondary subsystem within the same master container.

The present invention is also an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a liquid that is immiscible with water and has a positive coefficient of thermal expansion that is greater in absolute magnitude than the coefficient of thermal expansion of water; means for monitoring and controlling temperature of the master container; and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a liquid that is immiscible with water and has a negative coefficient of thermal expansion that is greater in absolute magnitude than the coefficient of thermal expansion of water; means for monitoring and controlling temperature of the master container, and means external to the master container for monitoring pressure inside of the master container.

In an alternate embodiment, the present invention is an apparatus for reducing excess pressure in isochoric systems comprising: a rigid and scalable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a liquid that is immiscible with water and has a positive coefficient of thermal expansion that is lesser in absolute magnitude than the coefficient of thermal expansion of water; means for monitoring and controlling temperature of the master container; and means external to the master container for monitoring pressure inside of the master container.

In an alternate embodiment, the present invention is an apparatus for reducing excess pressure in isochoric systems comprising: a rigid and scalable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a liquid that is immiscible with water and has a negative coefficient of thermal expansion that is lesser in absolute magnitude than the coefficient of thermal expansion of water; means for monitoring and controlling temperature of the master container; and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a solid that is immiscible with water and has a positive coefficient of thermal expansion that is greater in absolute magnitude than the coefficient of thermal expansion of water; means for monitoring and controlling temperature of the master container; and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a solid that is immiscible with water and has a negative coefficient of thermal expansion that is greater in absolute magnitude than the coefficient of thermal expansion of water; means for monitoring and controlling temperature of the master container; and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a solid that is immiscible with water and has a positive coefficient of thermal expansion that is lesser in absolute magnitude than the coefficient of thermal expansion of water; means for monitoring and controlling temperature of the master container; and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; and wherein the secondary subsystem is a solid that is immiscible with water and has a negative coefficient of thermal expansion that is lesser in absolute magnitude than the coefficient of thermal expansion of water, means for monitoring and controlling temperature of the master container, and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; wherein the secondary subsystem is a liquid that is miscible with water and has a positive coefficient of thermal expansion that is greater in absolute magnitude than the coefficient of thermal expansion of water; and wherein the liquid is separated from the primary subsystem by a mass-impermeable barrier; means for monitoring and controlling temperature of the master container; and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; wherein the secondary subsystem is a liquid that is miscible with water and has a negative coefficient of thermal expansion that is greater in absolute magnitude than the coefficient of thermal expansion of water; and wherein the liquid is separated from the primary subsystem by a mass-impermeable barrier; means for monitoring and controlling temperature of the master container; and means external to the master container for monitoring pressure inside of the master container.

In an alternate embodiment, the present invention is an apparatus for reducing excess pressure in isochoric systems comprising: a rigid and sealable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; wherein the secondary subsystem is a liquid that is miscible with water and has a positive coefficient of thermal expansion that is lesser in absolute magnitude than the coefficient of thermal expansion of water; and wherein the liquid is separated from the primary subsystem by a mass-impermeable barrier; means for monitoring and controlling temperature of the master container; and means external to the master container for monitoring pressure inside of the master container.

In an alternate embodiment, the present invention is an apparatus for reducing excess pressure in isochoric systems comprising: a rigid and scalable master container; wherein the master container has a volume; and wherein any bulk gas phase in the master container comprises less than five percent of the volume of the master container; a primary subsystem comprised of biological matter that is contained within the master container; a secondary subsystem that is contained within the master container; wherein water has a negative coefficient of thermal expansion at sub-0° Centigrade temperatures; wherein the secondary subsystem is a liquid that is miscible with water and has a negative coefficient of thermal expansion that is lesser in absolute magnitude than the coefficient of thermal expansion of water; and wherein the liquid is separated from the primary subsystem by a mass-impermeable barrier; means for monitoring and controlling temperature of the master container; and means external to the master container for monitoring pressure inside of the master container.

### BRIEF DESCRIPTION OF THE DRAWINGS

FIG. 1 is a flow chart that illustrates the steps of a preferred embodiment of the method of the present invention.

FIG. 2 is a continuation of FIG. 1.

FIG. 3 is a section view schematic illustrating the core components of a preferred embodiment of the apparatus of the present invention.

FIG. 4 is a graphical plot illustrating how various ratios of oil and water can alter the pressure-temperature relationship of an isochoric system.

### DETAILED DESCRIPTION OF INVENTION

### A. Overview

Conventional preservation by isochoric vitrification or isochoric supercooling involves: placing biological matter and a surrounding aqueous solution into a rigid container that does not transmit pressure (i.e., is rigid) or mass (i.e., is impermeable); removing excess air from the system; sealing the container such that the system is no longer in contact with the atmosphere or any other reservoir of pressure; and monitoring the temperature and pressure within the chamber. Such rigid containers are commonly used for preservation by isochoric freezing [8], [10], isochoric supercooling [11], and isochoric vitrification [4], and the pressure read inside the system for a given temperature indicates whether ice nucleation has occurred (and if so, to what degree).

In current methods and devices for preservation by isochoric supercooling or isochoric vitrification, the pressures to which the biological matter is exposed during cooling, warming, and storage are dictated by the thermo-volumetric properties (i.e., the thermal expansivity and the compressibility) of the solution that houses the biological matter, and these pressures may prove excessive and deleterious to the biological matter.

The present invention aims to reduce the excess positive or negative pressure to which biological matter is exposed during isochoric preservation by adding one or more subsystems to the interior contents of the isochoric chamber, the thermo-volumetric properties of which compensate the thermo-volumetric properties of the biological matter itself and/or the aqueous substance in which it is stored, resulting in a net reduction in the thermo-volumetric change of the system at the desired storage temperature, and thus a reduction in the excess pressure produced.

Specifically, if the biological matter and/or the solution in which it is housed, referred to as the “primary subsystem,” expands at the desired storage temperature, producing a positive excess pressure, the added subsystem, referred to as the “secondary subsystem,” will contract at the desired storage temperature, compensating the primary subsystem and reducing the ultimate positive pressure to which the biological matter is exposed. Similarly, if the primary subsystem contracts at the desired storage temperature, the secondary subsystem will expand, reducing the ultimate negative pressure to which the biological matter is exposed. The secondary subsystem does not contain biological matter and is either comprised of a material immiscible with aqueous solutions or includes a mass-impermeable barrier, such that it does not significantly alter the chemical composition of the primary sub-system.

The present invention reduces the excess positive or negative pressure to which biological matter is exposed during isochoric preservation by reducing the net thermo-volumetric change within the isochoric container at a given desired storage temperature.

### B. Detailed Description of the Figures

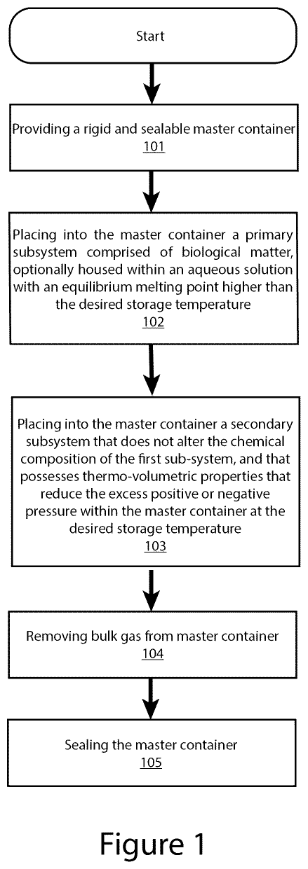

FIG. 1 is a flow chart that illustrates the initial steps of a preferred embodiment of the method of the present invention. First, a rigid and sealable master container is provided 101. Preferably, this container is constructed of an anodized aluminum alloy, a titanium alloy, a stainless steel alloy, or another metallic substance with high strength and high corrosion resistance. Next, a primary subsystem comprised of biological matter optionally contained within an aqueous solution with an equilibrium melting point higher than the desired storage temperature is placed within the master container 102. Next, a secondary subsystem is placed into the master container. This secondary subsystem has the following qualities: it does not substantially alter the chemical composition of the primary subsystem; it does not contain biological matter; and it possesses thermo-volumetric properties that compensate those of the primary subsystem in order to reduce the excess positive or negative pressure within the master container at the desired storage temperature 103. Preferably, this secondary subsystem is comprised of a liquid that is immiscible with water and has a positive coefficient of thermal expansion greater in absolute magnitude than the coefficient of thermal expansion of water, which is negative at sub-0 Centigrade temperatures. Next, all or most of the bulk gas phase is removed from the master container 104. Next, the master container is sealed 105.

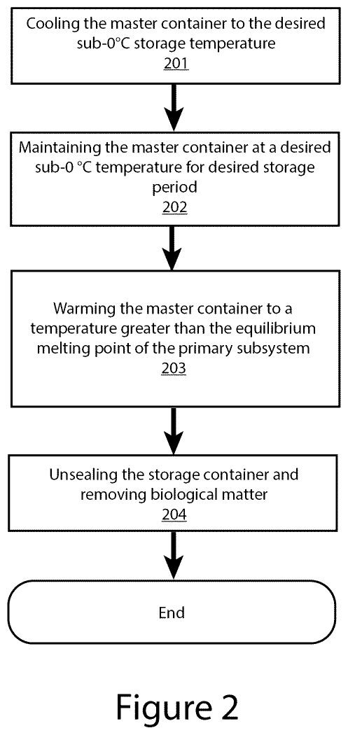

Referring to FIG. 2, the master container is cooled to a desired sub-0 Centigrade storage temperature 201. Next, the master container is maintained at this desired storage temperature for a desired storage period 202. Next, the master container is warmed to a temperature greater than the equilibrium melting point of the primary subsystem 203. Finally, the master container is unsealed, and the biological matter is removed 204.

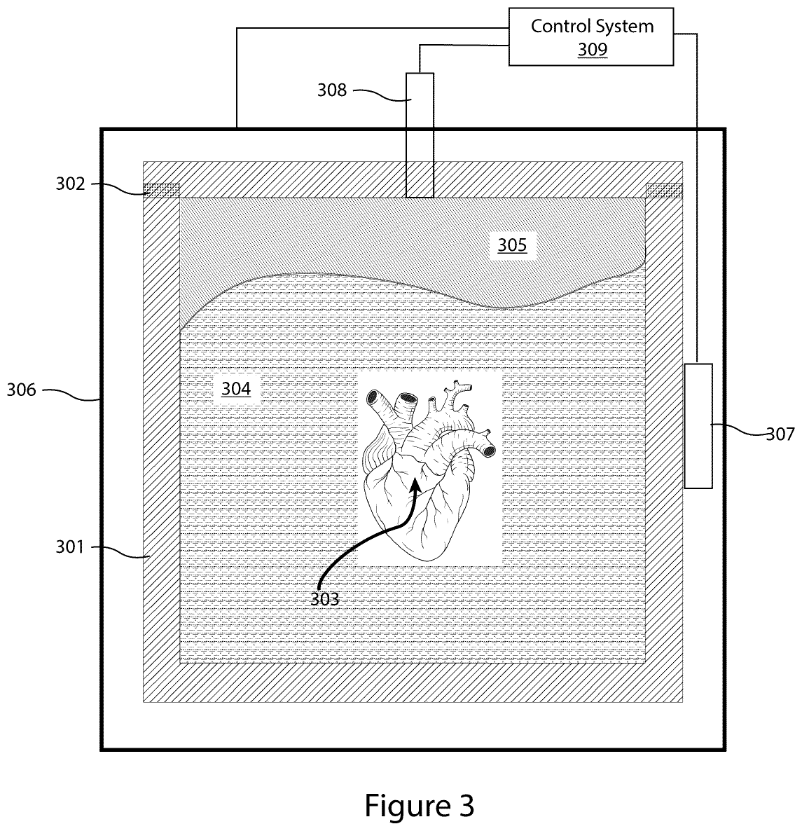

FIG. 3 is a section view schematic illustrating the core components of a preferred embodiment of the apparatus of the present invention. The apparatus comprises: a master container 301 that is rigid and has a seal 302 that can provide air- and liquid-tight sealing; a primary subsystem comprised of biological matter 303 optionally housed in an aqueous solution 304 that has an equilibrium melting point above the desired sub-zero centigrade storage temperature; and a secondary subsystem 305 comprised of a liquid or solid that does not substantially alter the chemical composition of the biological matter, that does not itself include biological matter, and the thermo-volumetric properties of which compensate those of the biological matter 303 and/or its housing aqueous solution 304, thereby minimizing the excess positive or negative pressure at the desired temperature.

In a preferred embodiment, the aqueous solution 304 in the primary subsystem is comprised of a dilute saline solution, as typically used in the preservation of organs or tissues, and the secondary subsystem 305 is comprised of mineral oil, perfluorocarbon, or another liquid immiscible with water. Dilute saline solutions, like pure water, will expand as they cool in the sub-0° C. temperature regime, which will generate excess positive pressure in a closed isochoric system, as provided by the master container 301. Mineral oil, perfluorocarbon, and nearly all other water-immiscible liquids will contract at these same temperatures, compensating the expansion of the saline and reducing excess pressure, to the benefit of the biological matter 303 stored within.

In an alternate embodiment, the aqueous solution 304 in the primary subsystem is comprised of an aqueous solution with a high concentration of cryoprotective additives (such as dimethyl sulfoxide, ethylene glycol, glycerol, propylene glycol, etc.). The aqueous solution 304 will contract significantly upon cooling, generating excess negative pressure within the master container 301. The secondary subsystem 305 may be comprised of pure water or a dilute solution contained within a mass-impermeable barrier. Pure water and dilute solutions thereof will expand upon cooling in the sub-0° C. temperature range, compensating the contractile aqueous solution 304 in the primary subsystem and reducing excess negative pressure. The mass-impermeable barrier will prevent chemical interaction between the secondary subsystem 305, on the one hand, and the aqueous solution 304 and the biological matter 303 in the primary subsystem, on the other hand.

Optionally, the apparatus may include an external means of providing temperature control and cooling/warming to the system 306, such as a bath of circulating liquid, gas, or vapor, a refrigerator, a phase-change material, a thermoelectric or Peltier module, a Stirling cooler, or a resistance heater, a means of monitoring the temperature of the system 307, such as a thermocouple, resistor, or thermometer; a means of monitoring the pressure within the outer container 308, such as a digital pressure transducer, a pressure gauge, a pressure-sensitive optical port, or a strain gauge; and a control system 309 such as a computer or microprocessor, which is in communication with the means of temperature and/or pressure measurement and the means of temperature control and cooling/warming.

The biological matter 303 to be preserved is often housed within an aqueous solution 304, i.e., a solution in which water acts as the solvent. In some embodiments of the invention, this solution 304 is comprised of water with or without added organic molecules or chemical cryoprotectants. These additives may dictate the range of temperatures to which the system can be supercooled without ice nucleation, or they may increase the stability of supercooling at a given preservation temperature. They may also increase the glass transition temperature of the solution to increase ease of vitrification, reduce the melting or freezing point of the solution, and/or minimize toxicity to the biological matter 303. These additives may also affect the thermo-volumetric properties of the water, decreasing or increasing the degree to which the primary subsystem expands or contracts at a given temperature, and/or decreasing or increasing the pressure generated from said expansion or contraction. Such chemical additives include, but are not limited to, dimethyl sulfoxide, ethylene glycol, polyethylene glycol, 3-OMG, glycerol, antifreeze proteins, ice recrystallization inhibitors, synthetic or organic ice modulators, sugars, sugar alcohols, amino acids, salts, etc.

By way of illustration but not limitation, the biological matter 303 may be comprised of human or non-human cells, organic molecules, multicellular constructs, tissues, organs, full organisms and/or food(s), including but not limited to stem cells, blood, bone marrow, blood vessels, pancreatic islets, reproductive tissues, skin, etc.; hearts, livers, kidneys, lungs, pancreases, spleens, etc.; eyes, full or partial limbs, fingers or toes, brains, spinal columns, dorsal ganglia, nervous tissue, etc.; engineered tissues such as 3D microtissue constructs, liver-on-a-chip constructs, lung-on-a-chip constructs, heart-on-a-chip constructs, etc.; full organisms such as zebrafish, coral, nematodes, or other marine or land-dwelling animals; and/or foodstuffs such as cherries, berries, potatoes, tomatoes, fish, beef, etc.

The biological matter 303 may be perfused with or in the aqueous solution 304 prior to preservation. The biological matter may also undergo some manner of conditioning prior to preservation, including, but not limited to, normothermic or hypothermic machine perfusion, passive or active perfusion with a liquid, or immersion in a liquid of any kind.

In some embodiments, multiple separate and/or different primary subsystems, comprised of biological matter 303 and optionally an aqueous solution 304 in which the biological matter is housed, may be added to the master container 301. Multiple separate and/or different secondary subsystems 305 may also be added, all of which may have different thermo-volumetric properties. The volume-averaged thermo-volumetric properties of the primary subsystems will be compensated by the volume-averaged thermo-volumetric properties of the secondary subsystems.

For example, in one embodiment, the master container 301 may contain both a human heart 303 housed in a dilute saline solution 304 of one composition and a human brain 303 contained in a dilute solution 304 of another composition. Both of the aqueous solutions 304, being dilute aqueous solutions, will expand upon cooling, generating unwanted excess positive pressure. The total excess pressure generated will be a function of the combined net expansion of the two primary subsystems. Added to the master container 301 may also be two secondary subsystems, one comprised of mineral oil and the other comprised of perlfuorocarbon, both of which contract upon cooling, compensating the expansion of the primary subsystems and reducing the excess positive pressure. The total excess pressure reduction will be a function of the combined net contraction of the two secondary subsystems 305. The final pressure in the master container 301 at the desired storage temperature will be a function of the net thermo-volumetric change of all of the subsystems, primary and secondary, combined.

The secondary subsystem(s) 305 may be comprised of a liquid substance that is generally immiscible with water, including hydrocarbons such as mineral oil, vegetable oil, or silicone oil; perfluorocarbons such as perfluorodecalin, perfluorotributylamine, perfluorooctyl bromide; or any other immiscible liquid. The secondary subsystem(s) 305 may also be comprised of a liquid substance that is miscible with water but is protected from mixing with the biological matter and/or its housing aqueous solution by a mass-impermeable membrane or container. The secondary subsystem(s) 305 may also be comprised of a solid, such as a rubber or plastic. Polymeric substances in particular, such as ABS plastic, nylon, etc., are known to contract significantly with temperature and may be used to compensate expansion of the primary subsystem.

The master container 301 and all contents within it may be stored for any amount of time at one or multiple temperatures between 0° C. and −273° C. 302 and may be cooled 201 and/or warmed 203 at any rate. In some embodiments, when the biological matter 303 to be stored is a human organ, an isochoric supercooling approach may be used, for which the desired storage temperature may be in the range 0° C. to −20° C. to ensure avoidance of nucleation from the supercooled state, and the desired cooling 201 and warming 203 rates may be between 0.01° C./min and 10° C./min so as to not avoid damage from excessively fast temperature change. In other embodiments, when the biological matter 303 to be stored is cells, reproductive matter such as sperm, oocytes, or embryos, or organisms such as coral, an isochoric vitrification approach may be used, for which the desired storage temperature may be in the range of −80° C. to −196° C. to facilitate the glass transition process, and the desired cooling and warming rates may be between 1° C./min and 1000° C./min to ensure avoidance of ice nucleation during the vitrification process.

In the preferred embodiment of the apparatus shown in FIG. 3, the master container 301 is cooled by a cooling and/or warming system 306 external to the outer container; however, an internal cooling and/or warming system may also be used, examples of which include internal heat exchanger pipes or internal phase-change materials. In all cases, the cooling and/or warming system 306 that modulates the temperature of the outer container 301 may be active (i.e., requiring an input of thermodynamic work), as in a refrigerator or circulating bath, or passive (i.e., proceeding spontaneously), as in a phase change material such as ice or a eutectic salt.

The master container 301 may be equipped with an implement to measure or infer the pressure within 308, such as a pressure transducer, a pressure gauge, a pressure-sensitive optical port, or a strain gage. This implement can be used to monitor the pressure either continuously or at discrete points in the process of cooling 201, storage 202, or warming 203. This pressure may be used to verify the successful reduction of excess pressure, and in the event that the pressure is higher or lower than anticipated or desired, the control system 309 communicating with the means of temperature control 306 and the pressure measurement implement 308 may enact changes in temperature based on such a reading from the pressure measurement implement. For example, when the biological matter 303 within the apparatus is a human heart intended for transplantation, and this heart is being stored 207 at a temperature of −4° C., if the pressure read were higher than atmospheric pressure (0.1 MPa), the control system 309 would issue a command to the temperature control implement 306 to warm the system by an increment less than 1° C., in order to reduce this excess pressure while maintaining a tenable supercooled storage temperature. The control system 309 may also be used to change or adjust the temperature of the system in response to any changes in the measured or inferred pressure within the system because, in isochoric systems, the temperatures and pressure are coupled.

In one embodiment, the master container 301 may also be instrumented with a piston, threaded rod, or another mechanical element that is configured to increase or decrease the volume of the master container. This element provides a supplementary measure by which to alter the pressure within the master container and may be used in combination with one or multiple secondary subsystems 305 to provide finer tuning of the pressure during preservation.

The master container 301 may feature additional measures to protect the contents within from vibration, including a sleeve, coating, mount, or other external feature made of a vibration-reducing material such as neoprene or other rubbers; springs or other mechanical features for vibration reduction; and/or combinations thereof. Vibration, which may be encountered during flight, ground-transport, or general use, may cause unwanted fluctuations in pressure in supercooled isochoric systems.

Undesired or uncontrolled changes in temperature can negatively affect stored biological matter 303, and can generate unwanted fluctuations in pressure, due to the temperature-pressure coupling inherent to isochoric systems. The master container 301 may thus feature additional measures to protect the stored supercooled or vitrified biological matter 303 from undesired temperature changes, including, but not limited to, a thermally insulating sheath, sleeve, or coating; a surrounding phase-change material; a vacuum-insulated panel, material, or chamber; and/or other thermal insulation measures.

The master container 301, the primary subsystem comprised of biological matter 203 and optionally an aqueous solution in which it is housed 304, and secondary subsystem 305 each may contain or be comprised of any volume, and a wide range of volumes may be desired based on the biological matter 303 to be stored or the excess pressure to be reduced. For example, to preserve mesenchymal stem cells by isochoric vitrification, the master container 301 may contain volumes in the 1 microliter to 10 mL range. By contrast, to preserve a human liver by isochoric supercooling, the master container may contain volumes in the 1 L-20 L range. In bulk agricultural applications, especially those intended for preservation of food during shipping, master containers 201 on the scale of 20-1000 L may be desired.

The master container 301 may also be made in full or in part of a transparent rigid material such as polycarbonate or sapphire, which may be used to study or monitor the internal contents or behaviors of the container during cooling 201, storage 202, or warming 203 of the system, including, but not limited to, the behavior of preserved biologics or of any phase transitions that may occur. In some embodiments, a fully or partially transparent master container 301 is integrated into a microscope platform, allowing microscopic examination of the contents contained therein. The container may also be constructed in geometries at the millimeter- or micron-length scale for these purposes.

The master container 301, although generally assumed not to participate substantially in the thermo-volumetric contraction/expansion process that dictates the pressure within, may also be constructed specifically to have a substantial thermo-volumetric effect. To achieve this end, the container may be constructed from a material with a high coefficient of thermal expansion relative to stainless steel or titanium, such as ABS plastic, or may be constructed from a geometry chosen to maximize contractile or expansive effects in a given direction, such as a cylinder, the contraction/expansion of which acts principally in the radial direction. This may be particularly preferable when the primary subsystem, comprised of biological matter 303 optionally stored within an aqueous solution 304, is highly contractile (as is the case when the aqueous solution 304 is highly concentrated). In this case, additional contraction of the master container 301 itself around the primary subsystem can aid in reducing excess negative pressure, complementing the reduction provided by the secondary subsystem 305.

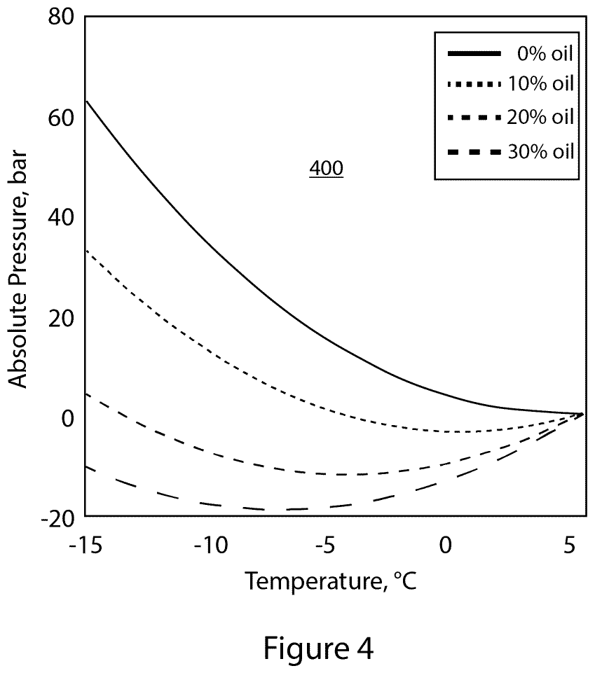

FIG. 4 is a graphical plot 400 illustrating how, using known thermo-volumetric properties of the primary and secondary subsystems and applying the simple principle of conservation-of-volume, the excess pressure generated at a given temperature may be calculated for differing ratios of the volumes of the two subsystems. Such calculation enables rational design of isochoric protocols to maximally reduce excess pressure. In this figure, the primary subsystem is approximated as having the properties of water (a reasonable approximation for most biological matter 303 and dilute aqueous solutions 304), and the secondary subsystem is approximated as having the properties of mineral oil. The figure legend gives the resulting pressure as a function of temperature for different volume percentages of the secondary subsystem.

### C. Example

In order to test the present invention, an apparatus was produced according to the general design of FIG. 3 and tested in preservation of biological matter by isochoric supercooling. In this example, the preserved biological model 303 was a rodent heart, which was stored 202 in two supercooled configurations for 24 hours at −4 C without ice nucleation.

The first configuration involved a single-phase isochoric system without the excess pressure reduction provided by the present invention, and the second configuration involved an isochoric system with a secondary subsystem 305 comprised of a volume of mineral oil. The first configuration without pressure control resulted in increased hydrostatic pressure of 61 bar at −4° C. due to the uncompensated expansion of the aqueous storage solution 304 (the University of Wisconsin solution, an approximately 300 mM dilute aqueous organ preservation solution, which is a clinical standard).

The second configuration, pursuant to the method and apparatus disclosed herein, incorporated a secondary subsystem 305 comprised of mineral oil. Mineral oil is immiscible with aqueous solutions and thereby does not alter the chemical composition of the biological matter 303 or its aqueous solution 304. Mineral oil also contracts with decreasing temperature in the sub-0 Centigrade range, counteracting the expansion of the University of Wisconsin solution and the heart itself. According to the basic estimates plotted in FIG. 4 400, the secondary subsystem 305 was chosen to occupy 21% of the initial volume of the master container 301, providing maximum reduction of excess pressure. At the desired storage temperature of −4° C., the pressure was 1 bar, identical to standard atmospheric conditions.

After 24 hours storage at −4° C. 202, rewarming 203, and removal of the rodent hearts from the chamber 204, the hearts were assessed on a Langendroff perfusion device using various functional metrics such as heart rate, left ventricular developed pressure (LVDP), rate-pressure product (RPP), and contraction/relaxation rate. A reduced heart rate, LVDP, RPP, and contraction/relaxation rate were measured for the heart stored in the conventional isochoric system without pressure modulation. On the other hand, the heart stored in the isochoric system with pressure control exhibited normal cardiac function.

### REFERENCES

[1] S. Giwa et al., “The promise of organ and tissue preservation to transform medicine,” Nat. Biotechnol., vol. 35, no. 6, pp. 530-542, 2017, doi: 10.1038/nbt.3889.
[2] M. J. Powell-Palm, A. Koh-Bell. and B. Rubinsky. “Isochoric conditions enhance stability of metastable supercooled water,” Appl. Phys. Lett., vol. 116, no. 12, 2020, doi: 10.1063/1.5145334.
[3] A. N. Consiglio, D. Lilley, R. Prasher, B. Rubinsky, and M. J. Powell-Palm, “Methods to stabilize aqueous supercooling identified by use of an isochoric nucleation detection (INDe) device,” Cryobiology, 2022, doi: https://doi.org/10.1016/j.cryobiol.2022.03.003.
[4] Y. Zhang et al., “Isochoric vitrification: An experimental study to establish proof of concept,” Cryobiology, 2018, doi: 10.1016/j.cryobiol.2018.06.005.
[5] G. M. Fahy, D. R. MacFarlane, C. A. Angell, and H. T. Meryman, “Vitrification as an approach to cryopreservation,” Cryobiology, vol. 21, no. 4, pp. 407-426, August 1984, doi: 10.1016/0011-2240(84)90079-8.
[6] B. Rubinsky, P. A. Perez, and M. E. Carlson, “The thermodynamic principles of isochoric cryopreservation,” Cryobiology, 2005, doi: 10.1016/j.cryobiol.2004.12.002.
[7] B. Chang et al., “On the pressure dependence of salty aqueous eutectics,” Cell Rep. Phys. Sci., p. 100856, 2022, doi: https://doi.org/10.1016/j.xcrp.2022.100856.
[8] L. Wan et al., “Preservation of rat hearts in subfreezing temperature isochoric conditions to −8° C. and 78 MPa,” Biochem. Biophys. Res. Commun., vol. 496, no. 3, pp. 852-857, February 2018, doi: 10.1016/j.bbrc.2018.01.140.
[9] L. Wan, M. J. Powell-Palm, M. G. Clemens, and B. Rubinsky, “Time-Dependent Effects of Pressure During Preservation of Rat Hearts in an Isochoric System at Subfreezing Temperatures.” Cryoletters, vol. 40, no. 1, pp. 64-70, January 2019.
[10]M. J. Powell-Palm. Y. Zhang, J. Aruda, and B. Rubinsky, “Isochoric conditions enable high subfreezing temperature pancreatic islet preservation without osmotic cryoprotective agents,” Cryobiology, vol. 86, pp. 130-133, February 2019. doi: 10.1016/j.cryobiol.2019.01.003.
[11]“Isochoric supercooled preservation and revival of human cardiac microtissues|Communications Biology.” https://www.nature.com/articles/s42003-021-02650-9 (accessed Jul. 11, 2023).

We claim:

1. A method for reducing excess pressure in isochoric systems comprising:
(a) providing a rigid and scalable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a liquid that is immiscible with water and has a positive coefficient of thermal expansion that is greater in absolute magnitude than the coefficient of thermal expansion of water;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

2. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a liquid that is immiscible with water and has a negative coefficient of thermal expansion that is greater in absolute magnitude than the coefficient of thermal expansion of water;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

3. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a liquid that is immiscible with water and has a positive coefficient of thermal expansion that is lesser in absolute magnitude than the coefficient of thermal expansion of water;
(d) removing bulk gas phase from the master container;
(e) scaling the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

4. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a liquid that is immiscible with water and has a negative coefficient of thermal expansion that is lesser in absolute magnitude than the coefficient of thermal expansion of water;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

5. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a solid that is immiscible with water and has a positive coefficient of thermal expansion that is greater in absolute magnitude than the coefficient of thermal expansion of water;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

6. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a solid that is immiscible with water and has a negative coefficient of thermal expansion that is greater in absolute magnitude than the coefficient of thermal expansion of water;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

7. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a solid that is immiscible with water and has a positive coefficient of thermal expansion that is lesser in absolute magnitude than the coefficient of thermal expansion of water;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

8. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures; and
wherein the secondary subsystem is a solid that is immiscible with water and has a negative coefficient of thermal expansion that is lesser in absolute magnitude than the coefficient of thermal expansion of water;
(d) removing bulk gas phase from the master container;
(e) sealing the master container;
(f) cooling the master container to a desired sub-0° Centigrade storage temperature:
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

9. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures;
wherein the secondary subsystem is a liquid that is miscible with water and has a positive coefficient of thermal expansion that is greater in absolute magnitude than the coefficient of thermal expansion of water; and
wherein the liquid is separated from the primary subsystem by a mass-impermeable barrier;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

10. A method for reducing excess pressure in isochoric systems comprising:
(a) providing a rigid and sealable master container:
(b) placing a primary subsystem comprised of biological matter into the master container;
(c) placing a secondary subsystem into the master container;
wherein water has a negative coefficient of thermal expansion at sub-0° Centigrade temperatures;
wherein the secondary subsystem is a liquid that is immiscible with water and has a negative coefficient of thermal expansion that is greater in absolute magnitude than the coefficient of thermal expansion of water; and
wherein the liquid is separated from the primary subsystem by a mass-impermeable barrier;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

11. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures;
wherein the secondary subsystem is a liquid that is immiscible with water and has a positive coefficient of thermal expansion that is lesser in absolute magnitude than the coefficient of thermal expansion of water; and
wherein the liquid is separated from the primary subsystem by a mass-impermeable barrier;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

12. A method for reducing excess pressure in isochoric systems comprising:
(a) providing a rigid and sealable master container;
(b) placing a primary subsystem comprised of biological matter into the master container;
(c) placing a secondary subsystem into the master container;
wherein water has a negative coefficient of thermal expansion at sub-0° Centigrade temperatures;
wherein the secondary subsystem is a liquid that is immiscible with water and has a negative coefficient of thermal expansion that is lesser in absolute magnitude than the coefficient of thermal expansion of water; and
wherein the liquid is separated from the primary subsystem by a mass-impermeable barrier;
(d) removing bulk gas phase from the master container;
(e) sealing the master container;
(f) cooling the master container to a desired sub-0° Centigrade storage temperature;
(g) maintaining the master container at the desired storage temperature for a desired storage period;
(h) wherein the primary subsystem has an equilibrium melting point, warming the master container to a temperature that is greater than the equilibrium melting point of the primary subsystem;
(i) unsealing the master container; and
(j) removing the biological matter from the master container.

13. The method of claim 1, 2, 3, 4, 5, 6, 7, 8, 9, 10, 11 or 12, wherein the secondary subsystem is comprised of the group consisting of mineral oil, vegetable oil, silicone oil, and perfluorocarbon.

14. The method of claim 1, 2, 3, 4, 5, 6, 7, 8, 9, 10, 11 or 12, wherein the secondary subsystem is comprised of pure water.

15. The method of claim 1, 2, 3, 4, 5, 6, 7, 8, 9, 10, 11 or 12, further comprising the step of:
(k) providing a mechanical element that is configured to increase and decrease volume of the master container.

16. The method of claim 1, 2, 3, 4, 5, 6, 7, 9, 10, 11 or 12, wherein the master container is comprised of a material that possesses a coefficient of thermal expansion that is higher than that of grade 5 titanium.

17. The method of claim 1, 2, 3, 4, 5, 6, 7, 8, 9, 10, 11 or 12, further comprising the step of:
(k) combining at least one primary subsystem and more than one secondary subsystem within the same master container.

18. The method of claim 1, 2, 3, 4, 5, 6, 7, 8, 9, 10, 11 or 12, further comprising the step of:
(k) combining more than one primary subsystem and at least one secondary subsystem within the same master container.

19. The method of claim 1, 2, 3, 4, 5, 6, 7, 8, 9, 10, 11 or 12, further comprising the step of:
(k) combining more than one primary subsystem and more than one secondary subsystem within the same master container.

20. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures; and
wherein the secondary subsystem is a liquid that is immiscible with water and has a positive coefficient of thermal expansion that is greater in absolute magnitude than the coefficient of thermal expansion of water;
(d) means for monitoring and controlling temperature of the master container; and
(e) means external to the master container for monitoring pressure inside of the master container.

21. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures; and
wherein the secondary subsystem is a liquid that is immiscible with water and has a negative coefficient of thermal expansion that is greater in absolute magnitude than the coefficient of thermal expansion of water;
(d) means for monitoring and controlling temperature of the master container, and
(e) means external to the master container for monitoring pressure inside of the master container.

22. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures; and
wherein the secondary subsystem is a liquid that is immiscible with water and has a positive coefficient of thermal expansion that is lesser in absolute magnitude than the coefficient of thermal expansion of water;
(d) means for monitoring and controlling temperature of the master container; and
(e) means external to the master container for monitoring pressure inside of the master container.

23. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures; and
wherein the secondary subsystem is a liquid that is immiscible with water and has a negative coefficient of thermal expansion that is lesser in absolute magnitude than the coefficient of thermal expansion of water;
(d) means for monitoring and controlling temperature of the master container; and
(e) means external to the master container for monitoring pressure inside of the master container.

24. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures; and
wherein the secondary subsystem is a solid that is immiscible with water and has a positive coefficient of thermal expansion that is greater in absolute magnitude than the coefficient of thermal expansion of water;
(d) means for monitoring and controlling temperature of the master container; and
(e) means external to the master container for monitoring pressure inside of the master container.

25. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures; and
wherein the secondary subsystem is a solid that is immiscible with water and has a negative coefficient of thermal expansion that is greater in absolute magnitude than the coefficient of thermal expansion of water;
(d) means for monitoring and controlling temperature of the master container; and
(e) means external to the master container for monitoring pressure inside of the master container.

26. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container,
(c) a secondary subsystem that is contained within the master container;
wherein water has a negative coefficient of thermal expansion at sub-0° Centigrade temperatures; and
wherein the secondary subsystem is a solid that is immiscible with water and has a positive coefficient of thermal expansion that is lesser in absolute magnitude than the coefficient of thermal expansion of water;
(d) means for monitoring and controlling temperature of the master container; and
(e) means external to the master container for monitoring pressure inside of the master container.

27. An apparatus for reducing excess pressure in isochoric systems comprising:
(a) a rigid and scalable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures; and
wherein the secondary subsystem is a solid that is immiscible with water and has a negative coefficient of thermal expansion that is lesser in absolute magnitude than the coefficient of thermal expansion of water;
(d) means for monitoring and controlling temperature of the master container; and
(e) means external to the master container for monitoring pressure inside of the master container.

28. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures;
wherein the secondary subsystem is a liquid that is miscible with water and has a positive coefficient of thermal expansion that is greater in absolute magnitude than the coefficient of thermal expansion of water; and
wherein the liquid is separated from the primary subsystem by a mass-impermeable barrier;
(d) means for monitoring and controlling temperature of the master container; and
(e) means external to the master container for monitoring pressure inside of the master container.

29. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures;
wherein the secondary subsystem is a liquid that is miscible with water and has a negative coefficient of thermal expansion that is greater in absolute magnitude than the coefficient of thermal expansion of water; and
wherein the liquid is separated from the primary subsystem by a mass-impermeable barrier;
(d) means for monitoring and controlling temperature of the master container; and
(e) means external to the master container for monitoring pressure inside of the master container.

30. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures;
wherein the secondary subsystem is a liquid that is miscible with water and has a positive coefficient of thermal expansion that is lesser in absolute magnitude than the coefficient of thermal expansion of water; and
wherein the liquid is separated from the primary subsystem by a mass-impermeable barrier;
(d) means for monitoring and controlling temperature of the master container; and
(e) means external to the master container for monitoring pressure inside of the master container.

31. An apparatus for reducing excess pressure in isochoric systems comprising:
(a) a rigid and sealable master container;
wherein the master container has a volume; and
wherein any bulk gas phase in the master container comprises less than five percent of the volume of the master container;
(b) a primary subsystem comprised of biological matter that is contained within the master container;
(c) a secondary subsystem that is contained within the master container;
wherein water has a negative coefficient of thermal expansion at sub-0° Centigrade temperatures;
wherein the secondary subsystem is a liquid that is miscible with water and has a negative coefficient of thermal expansion that is lesser in absolute magnitude than the coefficient of thermal expansion of water; and
wherein the liquid is separated from the primary subsystem by a mass-impermeable barrier;
(d) means for monitoring and controlling temperature of the master container; and
(e) means external to the master container for monitoring pressure inside of the master container.
